(19) United States
(12) Patent Application Publication
(10) Pub. No.: US 2010/0113404 A1
(43) Pub. Date: May 6, 2010

(54) ANTI-ANGIOGENIC ACTIVITY OF 2-METHOXYESTRADIOL ANALOGS IN COMBINATION WITH ANTI-CANCER AGENTS

(76) Inventors: THERESA LAVALLEE, Rockville, MD (US); WILLIAM E. FOGLER, Rockville, MD (US); CAROLYN F. SIDOR, Chapel Hill, NC (US)

Correspondence Address: KING & SPALDING, 1180 PEACHTREE STREET , NE, ATLANTA, GA, 30309-3521, US

(21) Appl. No.: 12/262,318

(22) Filed: Oct. 31, 2008

(51) Int. Cl. A61K 31/565; A61P 35/00

(52) U.S. Cl. 514/171

(57) ABSTRACT

The present invention relates generally to methods and compositions for treating diseases characterized by abnormal cell proliferation and/or abnormal or undesirable angiogenesis by administering antiangiogenic agents in combination with anti-cancer agents. More specifically, the present invention relates to methods and compositions for treating diseases characterized by abnormal cell proliferation and/or abnormal or undesirable angiogenesis by administering 2-methoxyestradiol analogs, in combination with anti-cancer agents.

### CROSS-REFERENCE TO RELATED APPLICATIONS

This application claims the benefit of U.S. Provisional Application No. 61/984,632, filed Nov. 1, 2007, which is incorporated herein by reference in its entirety.

### FIELD OF THE INVENTION

The present invention relates generally to compositions comprising anti-angiogenic agents in combination with anti-cancer agents and methods of use. More specifically, the present invention relates to methods and compositions of administering analogs of 2-methoxyestradiol with anti-cancer agents. More particularly, the present invention relates to methods of treating diseases characterized by abnormal cell proliferation and/or abnormal or undesirable angiogenesis by administering analogs of 2-methoxyestradiol in combination with anti-cancer agents.

### BACKGROUND OF THE INVENTION

The direct targeting of tumor cells by cytotoxic agents has been the main therapeutic strategy against advanced human malignant tumors. This strategy has achieved limited success in curing most cancer types, often only achieving temporary remission at the expense of negative systemic side effects. Several solid epithelial tumors are not sensitive to chemotherapy and there is an increasing problem in the development of drug resistance in tumors that are initially responsive to chemotherapy (Braverman, Am. Intern. Med., (1993) 118:630-32 and Gasparini et al. The Breast, (1993) 2:27-32). In addition, there is a growing appreciation for the role the stroma, or non-tumor cells, play in determining the growth, proliferation and metastasis of a tumor. Angiogenesis, in particular, has been shown to play an important role in this regard.

Angiogenesis is the generation of new blood vessels into a tissue or organ. Under normal physiological conditions, humans and animals undergo angiogenesis only in very specific, restricted situations. For example, angiogenesis is normally observed in wound healing, fetal and embryonal development, and formation of the corpus luteum, endometrium and placenta.

Angiogenesis is controlled through a highly regulated system of angiogenic stimulators and inhibitors. The control of angiogenesis has been found to be altered in certain disease states and, in many cases, pathological damage associated with the diseases is related to uncontrolled angiogenesis. Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. Endothelial cells, lining the lumen of blood vessels, then protrude through the basement membrane. Angiogenic stimulants induce the endothelial cells to migrate through the eroded basement membrane. The migrating cells form a “sprout” off the parent blood vessel where the endothelial cells undergo mitosis and proliferate. The endothelial sprouts merge with each other to form capillary loops, creating a new blood vessel.

Persistent, unregulated angiogenesis occurs in many disease states, tumor metastases, and abnormal growth by endothelial cells. The diverse pathological disease states in which unregulated angiogenesis is present have been grouped together as angiogenic-dependent or angiogenic-associated diseases.

The hypothesis that tumor growth is angiogenesis-dependent was first proposed in 1971. (Folkman, New Eng. J. Med., (1971) 285:1182-86). In its simplest terms, this hypothesis states: “Once tumor ‘take’ has occurred, every increase in tumor cell population must be preceded by an increase in new capillaries converging on the tumor.” Tumor ‘take’ is currently understood to indicate a prevascular phase of tumor growth in which a population of tumor cells occupying a few cubic millimeters volume, and not exceeding a few million cells, can survive on existing host microvessels. Expansion of tumor volume beyond this phase requires the induction of new capillary blood vessels. For example, pulmonary micrometastases in the early prevascular phase in mice would be undetectable except by high power microscopy on histological sections.

Examples of the indirect evidence which support this concept include:
(1) The growth rate of tumors implanted in subcutaneous transparent chambers in mice is slow and linear before neovascularization, and rapid and nearly exponential after neovascularization. (Algire, et al., J. Nat. Cancer Inst., (1945) 6:73-85).
(2) Tumors grown in isolated perfused organs where blood vessels do not proliferate are limited to 1-2 mm³ but expand rapidly to >1000 times this volume when they are transplanted to mice and become neovascularized. (Folkman, et al., Annals of Surgery, (1966) 164:491-502).
(3) Tumor growth in the avascular cornea proceeds slowly and at a linear rate, but switches to exponential growth after neovascularization. (Gimbrone, Jr., et al., J. Nat. Cancer Inst., (1974) 52:421-27).
(4) Tumors suspended in the aqueous fluid of the anterior chamber of a rabbit eye remain viable, avascular, and limited in size to <1 mm³. Once they are implanted on the iris vascular bed, they become neovascularized and grow rapidly, reaching 16,000 times their original volume within 2 weeks. (Gimbrone, Jr., et al., J. Exp. Med., 136:261-76).
(5) When tumors are implanted on a chick embryo chorioallantoic membrane, they grow slowly during an avascular phase of >72 hours, but do not exceed a mean diameter of 0.93+0.29 mm. Rapid tumor expansion occurs within 24 hours after the onset of neovascularization, and by day 7 these vascularized tumors reach a mean diameter of 8.0+2.5 mm. (Knighton, British J. Cancer, (1977) 35:347-56).
(6) Vascular casts of metastases in a rabbit liver reveal heterogeneity in size of the metastases, but show a relatively uniform cut-off point for the size at which vascularization is present. Tumors are generally avascular up to 1 mm in diameter, but are neovascularized beyond that diameter. (Lien, et al., Surgery, (1970) 68:334-40).
(7) In transgenic mice that develop carcinomas in the beta cells of the pancreatic islets, pre-vascular hyperplastic islets are limited in size to <1 mm. At 6-7 weeks of age, 4-10% of the islets become neovascularized, and from these islets arise large vascularized tumors of more than 1000 times the volume of the pre-vascular islets. (Folkman, et al., Nature, (1989) 339:58-61).
(8) A specific antibody against VEGF (vascular endothelial growth factor) reduces microvessel density and causes “significant or dramatic” inhibition of growth of three human tumors which rely on VEGF as their sole mediator of angiogenesis (in nude mice). The antibody does not inhibit growth of the tumor cells in vitro. (Kim, et al., Nature, (1993) 362:841-44).
(9) Anti-bFGF monoclonal antibody causes 70% inhibition of growth of a mouse tumor which is dependent upon secretion of bFGF as its only mediator of angiogenesis. The antibody does not inhibit growth of the tumor cells in vitro. (Hori, et al., Cancer Res., 1991) 51:6180-84).
(10) Intraperitoneal injection of bFGF enhances growth of a primary tumor and its metastases by stimulating growth of capillary endothelial cells in the tumor. The tumor cells themselves lack receptors for bFGF, and bFGF is not a mitogen for the tumor cells in vitro. (Gross, et al., Proc. Am. Assoc. Cancer Res., (1990) 31:79).
(11) A specific angiogenesis inhibitor (AGM-1470) inhibits tumor growth and metastases in vivo, but is much less active in inhibiting tumor cell proliferation in vitro. It inhibits vascular endothelial cell proliferation half-maximally at 4 logs lower concentration than it inhibits tumor cell proliferation. (Ingber, et al., Nature, (1990) 48:555-57). There is also indirect clinical evidence that tumor growth is angiogenesis dependent.
(12) Human retinoblastomas that are metastatic to the vitreous develop into avascular spheroids that are restricted to less than 1 mm³ despite the fact that they are viable and incorporate ³H-thymidine (when removed from an enucleated eye and analyzed in vitro).
(13) Carcinoma of the ovary metastasizes to the peritoneal membrane as tiny avascular white seeds (1-3 mm³). These implants rarely grow larger until one or more of them become neovascularized.
(14) Intensity of neovascularization in breast cancer (Weidner, et al., New Eng. J. Med., (1991) 324:1-8; Weidner, et al., J Nat. Cancer Inst., (1992) 84:1875-87) and in prostate cancer (Weidner, et al., Am. J Pathol., (1993) 143(2):401-09) correlates highly with risk of future metastasis.
(15) Metastasis from human cutaneous melanoma is rare prior to neovascularization. The onset of neovascularization leads to increased thickness of the lesion and an increased risk of metastasis. (Srivastava, et al., Am. J. Pathol., (1988) 133:419-23).
(16) In bladder cancer, the urinary level of an angiogenic protein, bFGF, is a more sensitive indicator of status and extent of disease than is cytology. (Nguyen, et al., J. Nat. Cancer Inst., (1993) 85:241-42).

Thus, it is clear that angiogenesis plays a major role in the metastasis of cancer. If this angiogenic activity could be repressed or eliminated, then the tumor, although present, would not grow. In the disease state, prevention of angiogenesis could avert the damage caused by the invasion of the new microvascular system. Therapies directed at control of the angiogenic processes could lead to the abrogation or mitigation of these diseases.

Angiogenesis has been associated with a number of different types of cancer, including solid tumors and blood-borne tumors. Solid tumors with which angiogenesis has been associated include, but are not limited to, rhabdomyosarcomas, retinoblastoma, Ewing's sarcoma, neuroblastoma, and osteosarcoma. Angiogenesis is also associated with blood-borne tumors, such as leukemias, any of various acute or chronic neoplastic diseases of the bone marrow in which unrestrained proliferation of white blood cells occurs, usually accompanied by anemia, impaired blood clotting, and enlargement of the lymph nodes, liver and spleen. It is believed that angiogenesis plays a role in the abnormalities in the bone marrow that give rise to leukemia tumors and multiple myeloma diseases.

One of the most frequent angiogenic diseases of childhood is the hemangioma. A hemangioma is a tumor composed of newly formed blood vessels. In most cases the tumors are benign and regress without intervention. In more severe cases, the tumors progress to large cavernous and infiltrative forms and create clinical complications. Systemic forms of hemangiomas, hemangiomatoses, have a high mortality rate. Therapy-resistant hemangiomas exist that cannot be treated with therapeutics currently in use.

Angiogenesis is also responsible for damage found in heredity diseases such as Osler-Weber-Rendu disease, or heredity hemorrhagic telangiectasia. This is an inherited disease characterized by multiple small angiomas, tumors of blood or lymph vessels. The angiomas are found in the skin and mucous membranes, often accompanied by epitaxis (nose bleeds) or gastrointestinal bleeding and sometimes with pulmonary or hepatitic arteriovenous fistula.

Several compounds have been used to inhibit angiogenesis. Taylor et al. (Nature, (1982) 297:307) have used protamine to inhibit angiogenesis. The toxicity of protamine limits its practical use as a therapeutic. Folkman et al. (Science, (1983) 221:719, and U.S. Pat. Nos. 5,001,116 and 4,994,443) have disclosed the use of heparin and steroids to control angiogenesis. Steroids, such as tetrahydrocortisol, which lack glucocorticoid and mineralocorticoid activity, have been found to be angiogenic inhibitors.

Other factors found endogenously in animals, such as a 4 kDa glycoprotein from bovine vitreous humor and a cartilage derived factor, have been used to inhibit angiogenesis. Cellular factors, such as interferon, inhibit angiogenesis. For example, interferon alpha or human interferon beta have been shown to inhibit tumor-induced angiogenesis in mouse dermis stimulated by human neoplastic cells. Interferon beta is also a potent inhibitor of angiogenesis induced by allogeneic spleen cells. (Sidky, et al., Cancer Res., (1987) 47:5155-61). Human recombinant interferon (alpha/A) was reported to be successfully used in the treatment of pulmonary hemangiomatosis, an angiogenesis-induced disease. (White, et al., New Eng. J. Med., (1989) 320:1197-1200).

Other agents that have been used to inhibit angiogenesis include ascorbic acid ethers and related compounds. (Japanese Kokai Tokkyo Koho No.58-13 (1978)). Sulfated polysaccharide DS 4152 also inhibits angiogenesis. (Japanese Kokai Tokkyo Koho No. 63-119500). Additional anti-angiogenic compounds include Angiostatin® (U.S. Pat. Nos. 5,639,725; 5,792,845; 5,885,795; 5,733,876; 5,776,704; 5,837,682; 5,861,372, and 5,854,221) and Endostatin (U.S. Pat. No. 5,854,205).

Another compound which has been shown to inhibit angiogenesis is thalidomide. (D'Amato, et al., Proc. Natl. Acad. Sci., (1994) 90:4082-85). Thalidomide is a hypnosedative that has been successfully used to treat a number of diseases, such as rheumatoid arthritis (Gutierrez-Rodriguez, Arthritis Rheum., (1984) 27(10):1118-21; Gutierrez-Rodriguez, et al., J. Rheumatol., (1989) 16(2):158-63), and Behcet's disease (Handley et al., Br. J. Dermatol., 127 Suppl, (1992) 40:67-8; Gunzler, Med. Hypotheses, (1989) 30(2):105-9).

Although thalidomide has minimal side effects in adults, it is a potent teratogen. Thus, there are concerns regarding its use in women of child-bearing age. Although minimal, there are a number of side effects that limit the desirability of thalidomide as a treatment. One such side effect is drowsiness. In a number of therapeutic studies, the initial dosage of thalidomide had to be reduced because patients became lethargic and had difficulty functioning normally. Another side effect limiting the use of thalidomide is peripheral neuropathy, in which individuals suffer from numbness and dysfunction in their extremities.

Thus, improved methods and compositions are needed that are easily administered and capable of inhibiting angiogenesis. Additionally, what is needed are safe and effective treatments that cause minimal unwanted side effects.

2-Methoxyestradiol is a naturally occurring derivative of estradiol and has been shown to be an orally active, well-tolerated small molecule that possess anti-tumor and anti-angiogenic activity (Pribluda et al. Cancer Metastasis Rev. (2000) 19(1-2):173-9). 2-Methoxyestradiol has low affinity for estrogen receptors, α and β, and its antiproliferative activity is independent of the interaction with those receptors (LaVallee et al. Cancer Research (2002) 62(13):3691-7). Several mechanisms have been proposed for 2-methoxyestradiol activity, including those mediated by its ability to bind to the colchicines binding site of tubulin (Cushman et al., 1995; D'Amato et al., 1994), destabilization of microtubules and inhibition of HIF-1α nuclear accumulation (Mabjeesh et al. Cancer Cell, (2003) 3:363-375), induction of the extrinsic apoptotic pathway through upregulation of Death Receptor 5 (LaVallee et al. Cancer Research (2003) 63(2):469-75) and induction of the intrinsic apoptotic pathway, potentially through the inhibition of superoxide dismutase enzymatic activity (Huang et al. Trends Cell Biology (2001) 11(8):343-8).

2-Methoxyestradiol has been shown to inhibit multiple mechanisms of progression in myeloma cells in vitro and in vivo and the ability to evade resistance mechanisms implicated in clinical resistance to conventional therapies (Dingli et al. Clinical Cancer Research, (2002) 8:3984-3954). Inhibition of diverse tumor types including osteosarcoma (Maran et al. Bone, (2002) 30:393-398 and Golebiewska et al. Act Biochim Pol., (2002) 49:59-65), Ewing sarcoma (Djavaheri-Mergny et al. Oncogene, (2003) 22:2558-67), pancreatic adenoma (Qanungo et al. Oncogene, (2002) 21:4149-57 and Ryschich et al. Pancreas, (2003) 26:166-172), colon (Carothers et al. Cancer Lett., (2002) 187:77-86), medulloblastoma (Kumar et al. Carcinogenesis, (2003) 24:209-216), and melanoma (Ghosh et al. Melanoma Research, (2003) 13:119-127) has also been demonstrated with 2-methoxyestradiol. In vitro data suggests that 2-methoxyestradiol does not engage the estrogen receptor for its anti-proliferative activity and is not estrogenic over a wide range of concentrations, as assayed by estrogen dependent MCF-7 cell proliferation.

Anti-cancer therapy suffers from a number of limitations including development of drug resistance, unwanted systemic side effects, and limited efficacy against metastases. The use of combination therapy in order to overcome drug resistance, limit the unwanted side effects of certain anti-cancer agents, and improve their overall efficacy have been explored using various anti-cancer agent combinations. Finding a safe and effective combination has not been easy. Combining anti-cancer agents has often led to combined toxicities or formulations containing dosages of limited use or efficacy. Therefore, there is a need to find formulations and methods of administering anti-cancer agents that can be combined safely and effectively without having to resort to dose-reduction of either agent, or in the case where dose-reduction is necessary or desired, the combination maintains efficacy by allowing for higher doses of the less toxic agent. Analogs of 2-methoxyestradiol, with their multiple mechanisms of action, broad spectrum of inhibitory activity in a wide range of tumors, ability to overcome drug resistance in certain tumor types, limited negative side effects, and ability to prevent the formation of estrogenic metabolites make them strong candidates for combination therapy. Previous studies have not looked at the ability to combine analogs of 2-methoxyestradiol and anti-cancer agents into a method of treating diseases characterized by abnormal cell proliferation and/or abnormal or undesirable angiogenesis. What is needed therefore, are novel compositions and methods of treating diseases characterized by abnormal cell proliferation (i.e., abnormal mitosis) and/or abnormal or undesirable angiogenesis comprising combination therapy involving analogs of 2-methoxyestradiol together with one or more anti-cancer agents. Such compositions should be easy to administer and provide minimal or no side effects.

### SUMMARY OF THE INVENTION

The present invention comprises methods and compositions for treating disease characterized by abnormal cell proliferation and/or abnormal or undesirable angiogenesis comprising administering certain analogs of 2-methoxyestradiol in combination with anti-cancer agents. Specifically, the present invention relates to administering analogs of 2-methoxyestradiol that have been modified at the 2, 3, and 17 positions thereof, in combination with anti-cancer agents. Analogs of 2-methoxyestradiol within the general Formula I (shown below) that inhibit cell proliferation are preferred. Compounds within Formula I that inhibit angiogenesis are also preferred. Preferred analogs of 2-methoxyestradiol may also exhibit a change (increase or decrease) in estrogen receptor binding, or improved absorption, transport (e.g., through the blood-brain barrier and cellular membranes), biological stability, increased activity, or decreased toxicity.

A mammalian disease characterized by undesirable cell mitosis, as defined herein, includes, but is not limited to, excessive or abnormal stimulation of endothelial cells (e.g., atherosclerosis), solid tumors and tumor metastasis, benign tumors, for example, hemangiomas, acoustic neuromas, neurofibromas, trachomas, and pyogenic granulomas, vascular malfunctions, abnormal wound healing, inflammatory and immune disorders, Bechet's disease, gout or gouty arthritis, abnormal angiogenesis accompanying rheumatoid arthritis, skin diseases, such as psoriasis, diabetic retinopathy and other ocular angiogenic diseases such as retinopathy of prematurity (retrolental fibroplastic), macular degeneration, corneal graft rejection, neovascular glaucoma and Osler Weber syndrome (Osler-Weber-Rendu disease). Other undesired angiogenesis involves normal processes including ovulation and implantation of a blastula. Accordingly, the compositions described above can be used to block ovulation and implantation of a blastula or to block menstruation (induce amenorrhea).

It is known that 2-methoxyestradiol (2ME₂), an endogenous metabolite of estradiol with no intrinsic estrogenic activity, is an antiproliferative agent that induces apoptosis in a wide variety of tumor and non-tumor cell lines. When administered orally, it exhibits antitumor and antiangiogenic activity with little or no toxicity. Currently, 2ME₂ is in several clinical trials under the name PANZEM®(EntreMed, Inc., Rockville, Md.).

A novel series of compounds has been prepared that retains the biological activities of 2ME₂ but is believed to have reduced metabolism. Most of these analogs lack the hydroxyl moiety at position 17 and cannot be metabolized to 2-methoxyestrone or conjugated at that position. These 17-position analogs retain antiproliferative activity in HUVEC and tumor cells. Replacement of the 2-methoxy group by other moieties, such as an ethoxy or a propynyl group, retain antiproliferative activity, and these functionalities cannot be de-methylated to yield the estrogenic 2-hydroxyl derivatives. Also disclosed are compounds and methods for altering the chemical nature of position 3 in order to further prevent conversion to 2-methoxyestrone and/or the conjugation of 2-methoxyestradiol (or metabolites) with other molecules and subsequent loss during excretion of the resulting compounds.

Accordingly, it is an object of the present invention to provide a composition combining analogs of 2-methoxyestradiol in combination with one or more anti-cancer agents.

A further object of the present invention is to provide a method for administering analogs of 2-methoxyestradiol in combination with anti-cancer agents to mammals to more effectively inhibit angiogenesis and/or to more effectively treat diseases or conditions associated with undesirable and/or excessive angiogenesis and/or undesirable cell mitosis.

These and other objects, features and advantages of the present invention will become apparent after a review of the following detailed description of disclosed embodiments and the appended claims.

### DETAILED DESCRIPTION OF THE INVENTION

The present invention may be understood more readily by reference to the following detailed description of specific embodiments included herein. Although the present invention has been described with reference to specific details of certain embodiments thereof, it is not intended that such details should be regarded as limitations upon the scope of the invention. The entire text of the references mentioned herein are hereby incorporated in their entireties by reference.

The present invention comprises methods and compositions for treating diseases or conditions associated with or dependent on abnormal, undesirable and/or excessive angiogenesis and/or undesirable cell proliferation comprising administering to a human or animal an analog of 2-methoxyestradiol in combination with one or more anti-cancer agents.

Analogs of 2-methoxyestradiol

As described below, compounds that are useful in accordance with the invention include novel 2-methoxyestradiol derivatives that exhibit anti-mitotic, anti-angiogenic and/or anti-tumor properties. Preferred compounds of the invention are 2-methoxyestradiol derivatives modified at the 2-, 3- or 17-positions, or at combinations of the 2, 3 and 17 positions. Particularly preferred compounds are those of the general Formula I:

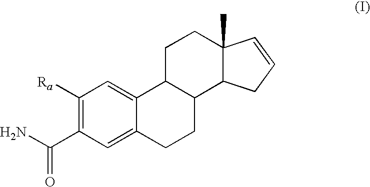

wherein Rₐ is selected from —OCH₃, —OCH₂CH₃ or —C≡C—CH₃. Preferred species according to the present invention are described below.

In an alternate disclosed embodiment of the present invention, compounds according to the present invention are those of Formula I, wherein Rₐ is —OCH₃.

In another alternate disclosed embodiment of the present invention, compounds according to the present invention are those of Formula I, wherein Rₐ is —OCH₂CH₃.

In a further alternate disclosed embodiment of the present invention, compounds according to the present invention are those of Formula I, wherein Rₐ is —C≡C—CH₃.

In each of the cases where stereoisomers are possible, both R and S stereoisomers are envisioned as well as any mixture of stereoisomers.

Those skilled in the art will appreciate that the invention extends to other compounds within the formula given in the claims below, having the described characteristics. These characteristics can be determined for each test compound using the assays detailed below and elsewhere in the literature.

### Anti-Proliferative Activity In Vitro

The process or processes by which 2ME₂ affects cell growth remains unclear, however, a number of studies have implicated various mechanisms of action and cellular targets. 2ME₂ induced changes in the levels and activities of various proteins involved in the progression of the cell cycle. These include cofactors of DNA replication and repair, e.g., proliferating cell nuclear antigen (PCNA) (Klauber, N., Parangi, S., Flynn, E., Hamel, E. and D'Amato, R. J. “Inhibition of angiogenesis and breast cancer in mice by the microtubule inhibitors 2-methoxyestradiol and Taxol,” Cancer Research, (1997) 57:81-86; Lottering, M-L., de Kock, M., Viljoen, T. C., Grobler, C. J. S. and Seegers, J. C. “17β-Estradiol metabolites affect some regulators of the MCF-7 cell cycle,” Cancer Letters, (1996) 110:181-186); cell division cycle kinases and regulators, e.g., p34^{cdc2} and cyclin B (Attalla, H., Mäkelä, T. P., Adlercreutz, H. and Andersson, L. C. “2-Methoxyestradiol arrests cells in mitosis without depolymerizing tubulin,” Biochemical and Biophysical Research Communications, (1996) 228:467-473; Zoubine, M. N., Weston, A. P., Johnson, D. C., Campbell, D. R. and Banerjee, S. K. “2-Methoxyestradiol-induced growth suppression and lethality in estrogen-responsive MCF-7 cells may be mediated by down regulation of p34cdc2 and cyclin B1 expression,” Int J Oncol., (1999) 15:639-646); transcription factor modulators, e.g., SAPK/JNK (Yue, T-L., Wang, X., Louden, C. S., Gupta, L. S., Pillarisetti, K., Gu, J-L., Hart, T. K., Lysko, P. G. and Feuerstein, G. Z. “2-Methoxyestradiol, an endogenous estrogen metabolite induces apoptosis in endothelial cells and inhibits angiogenesis: Possible role for stress-activated protein kinase signaling pathway and fas expression,” Molecular Pharmacology, (1997) 51:951-962; Attalla, H., Westberg, J. A., Andersson, L. C., Aldercreutz, H. and Makela, T. P. “2-Methoxyestradiol-induced phosphorylation of bcl-2: uncoupling from JNK/SAPK activation,” Biochem and Biophys Res Commun., (1998) 247:616-619); and regulators of cell arrest and apoptosis, e.g., tubulin (D'Amato, R. J., Lin, C. M., Flynn, E., Folkman, J. and Hamel, E. “2-Methoxyestradiol, and endogenous mammalian metabolite, inhibits tubulin polymerization by interacting at the colchicine site.,” Proc. Natl. Acad. Sci. USA, (1994) 91:3964-3968; Hamel, E., Lin, C. M., Flynn, E. and D'Amato, R. J. “Interactions of 2-methoxyestradiol, and endogenous mammalian metabolite, with unpolymerized tubulin and with tubulin polymers,” Biochemistry, (1996) 35:1304-1310), p21^{WAF1/CIP1} (Mukhopadhyay, T. and Roth, J. A. “Induction of apoptosis in human lung cancer cells after wild-type p53 activation by methoxyestradiol,” Oncogene, (1997) 14:379-384), bcl-2 and FAS (Yue et al. (1997); Attalla et al. (1998)), and p53 (Kataoka, M., Schumacher, G., Cristiano, R. J., Atkinson, E. N., Roth, J. A. and Mukhopadhyay, T. “An agent that increases tumor suppressor transgene product coupled with systemic transgene delivery inhibits growth of metastatic lung cancer in vivo,” Cancer Res., (1998) 58:4761-4765; Mukhopadhyay et al. (1997); Seegers, J. C., Lottering, M-L., Grobler C. J. S., van Papendorp, D. H., Habbersett, R. C., Shou, Y. and Lehnert B. E. “The mammalian metabolite, 2-methoxyestradiol, affects p53 levels and apoptosis induction in transformed cells but not in normal cells,” J. Steroid Biochem. Molec. Biol., (1997) 62:253-267). The effects on the level of cAMP, calmodulin activity and protein phosphorylation may also be related to each other. More recently, 2ME₂ was shown to upregulate Death Receptor 5 and caspase 8 in human endothelial and tumor cell lines (LaVallee T M, Zhan X H, Johnson M S, Herbstritt C J, Swartz G, Williams M S, Hembrough W A, Green S J, Pribluda V. S. “2-Methoxyestradiol up-regulates death receptor 5 and induces apoptosis through activation of the extrinsic pathway,” Cancer Res. (2003) 63(2):468-75). Additionally, 2ME₂ has been shown to interact with superoxide dismutase (SOD) 1 and SOD 2 and to inhibit their enzymatic activities (Huang, P., Feng, L., Oldham, E. A., Keating, M. J., and Plunkett, W. “Superoxide dismutase as a target for the selective killing of cancer cells,” Nature, (2000) 407:390-5). All cellular targets described above are not necessarily mutually exclusive to the inhibitory effects of 2ME₂ in actively dividing cells.

The high affinity binding of 2ME₂ to sex hormone-binding globulin (“SHBG”) has been mechanistically associated with its efficacy in a canine model of prostate cancer, in which signaling by estradiol and 5α-androstan-3α,17β-diol were inhibited by 2ME₂ (Ding, V. D., Moller, D. E., Feeney, W. P., Didolkar, V., Nakhla, A. M., Rhodes, L., Rosner, W. and Smith, R. G., “Sex hormone-binding globulin mediates prostate androgen receptor action via a novel signaling pathway,” Endocrinology, (1998) 139:213-218).

The more relevant mechanisms described above have been extensively discussed in Victor S. Pribluda, Theresa M. LaVallee and Shawn J. Green, 2-Methoxyestradiol: A novel endogenous chemotherapeutic and antiangiogenic in The New Angiotherapy, Tai-Ping Fan and Robert Auerbach eds., Human Press Publisher.

Assays relevant to these mechanisms of action and inhibition of cell proliferation are well-known in the art. For example, anti-mitotic activity mediated by effects on tubulin polymerization activity can be evaluated by testing the ability of a 2-methoxyestradiol analog to inhibit tubulin polymerization and microtubule assembly in vitro. Microtubule assembly can be followed in a Gilford recording spectrophotometer (model 250 or 2400S) equipped with electronic temperature controllers. A reaction mixture typically contains 1.0 M monosodium glutamate (pH 6.6), 1.0 mg/ml (10 μM) tubulin, 1.0 mM MgCl₂, 4% (v/v) dimethylsulfoxide and 20-75 μM of a composition to be tested. The reaction mixtures are incubated for 15 min. at 37° C. and then chilled on ice. After addition of 10 μl 2.5 mM GTP, the reaction mixture is transferred to a cuvette at 0° C., and a baseline established. At time zero, the temperature controller of the spectrophotometer is set at 37° C. Microtubule assembly is evaluated by increased turbidity at 350 nm. Alternatively, inhibition of microtubule assembly can be followed by transmission electron microscopy as described in Example 2 of U.S. Pat. Nos. 5,504,074, 5,661,143, and 5,892,069, the disclosures of which are incorporated herein by reference.

Other such assays include counting of cells in tissue culture plates or assessment of cell number through metabolic assays or incorporation into DNA of labeled (radiochemically, for example ³H-thymidine, or fluorescently labeled) or immuno-reactive (BrdU) nucleotides. In addition, antiangiogenic activity may be evaluated through endothelial cell migration, endothelial cell tubule formation, or vessel outgrowth in ex-vivo models such as rat aortic rings.

### Anti-Cancer Agents

Anti-cancer agents that may be used with the disclosed embodiment of the present invention include, but are not limited to, chemotherapeutics, angiogenesis inhibitors, kinase inhibitors, histone deacetylase inhibitors, as well as other modifiers of epigenetic phenomena and proteosome inhibitors.

### Chemotherapeutic Agents

Chemotherapeutic agents are those compounds that non-specifically target rapidly dividing cells. They include alkylating agents, antimetabolites, anti-tumor antibiotics, mitotic inhibitors and nitrosureas. Representative chemotherapeutic agents that may be used in the instant invention include, but are not limited to, the following; Aldeskeukin, Alemtuzumab, alitretinoin, allopurinol, altretamine, amifostine, anastrozole, arsenic trioxide, asparaginase, BCG Live, bexarotene capsules, bexarotene gel, bleomycin, busulfan intravenous, busulfan oral, calusterone, capecitabine, carboplatin, carmustine, carmustine with Polifeprosan 20 implant, celecoxib, chlorambucil, cisplatin, cladribine, cyclophosphamide, cytarabine, cytarabine liposomal, dacarbazine, dactinomycin actinomycin D, Darbepoetin alfa, daunorubicin liposomal, daunorubicin, daunomycin, Denileukin difitox, dexrazoxane, docetaxel, doxorubicin, doxorubicin liposomal, Dromostanolone propionate, Elliot's B solution® (Orphan Medical Inc. Minnetonka, Minn.), epirubicin, Epoetin alfa, estramustine, etoposide phosphate, etoposide VP-16, exemestane, Filgrastim, floxuridine, fludarabine, fluorouracil, fulvestrant, gemcitabine, bemtuzumab ozogamicin, goserelin acetate, hydroxyurea, Ibritumomab Tiuxetan, idarubicin, ifosfamide, imatinib mesylate, Interferon alfa-2a, Interferon alfa-2b, irinotecan, letrozole, leucovorin, levamisole, lomustine CCNU, meclorethamine (nitrogen mustard), megestrol acetate, melphalan (L-PAM), mercaptopurine (6-MP), mesna, methotrexate, methoxsalen, mitomycin C, mitotane, mitoxantrone, MKC-1 nadrolone phenpropionate, Nofetumomab, Oprelvekin, oxaliplatin, paclitaxel, pamidronate, pegademase, Pegaspargase, Pegfilgrastim, pnetostatin, pipobroman, plicamycin (mithramycin), porfimer sodium, procarbazine, quinacrine Rasburicase, Rituximab, Sargramotim, streptozocin, talc, tamoxifen, taxanes, temozolomide, teniposide (VM-26), testolactone, thioguanine (6-TG), thiotepa, topotecan, toremifene, Tositumomab, Trastuzumab, tretinoin (ATRA), Uracil Mustard, valrubicin, vinblastine, vincristine, vinorelbine and zoledronate.

### Angiogenesis Inhibitors

Angiogenesis inhibitors are those compounds that prevent the growth of new capillary blood vessels from preexisting blood vessels. Angiogenesis inhibitors that may be used in the present invention include both small-molecule and endogenous inhibitors of angiogenesis. Representative angiogenesis inhibitors that may be use in the present invention include, but are not limited to, alpha interferon, angiogenic steroids, anti-VEGFR inhibitors (Sunitinib, Sorafenib), Bevacizumab, Batimastat (BB-94), Carboxyaminoimidazole (CAI), CM101 (GBS toxin), CT-2548, hydrocortisone/beta-cyclodextran, interleukin-12, Linomide, Marimastat (BB-2516), Octreotide (somatostatin analogue), Pentosan polysulfate, platelet factor 4, Roquinimex (LS-2616, linomide), Suramin, SU101, Tecogalan sodium (DS-4152), thalidomide and its derivatives, TNP-470 (AGM-1470), angiostatin, endostatin, beta interferon, gamma interferon, cartilage-derived inhibitor (CDI), gamma interferon inducible protein (IP-10), gro-beta, heparinases, 2-methoxyestradiol, placental ribonuclease inhibitor, plasminogen activator inhibitor, proliferen-related protein, retinoids, thrombospondin, TIMP-2, and 16 kd prolactin.

### Kinase Inhibitors

Kinase inhibitors that may be used in the present invention include, but are not limited to, inhibitors of the following kinase families EGFR, HER2, VEGF receptors, FGF receptors, PDGF receptor, FLT3, ABL, SRC, Janus (JAK), MTOR, Rho, cyclin dependent kinases (CDK), protein kinase C (PKC), phosphatidylinositol-3-kinase (PI3K), Aurora, MAP/MEK, Jun N-terminal (JNK). Representative kinase inhibitors that may be used with the following invention include, but are not limited to, cetuximab, Erlotinib, gefitinib, PKI-166, Canertinib (CI-1033), SU-11464, Matuzumab (Emd7200), EKB-569, Zd6474 (Vandetanib), Trastuzumab, GW-572016 (lapatinib ditosylate), PKC-412 (Midostaurin), Vatalanib (Ptk787/ZK222584), CEP-701 (Lestaurtinib), SU5614, MLN518, XL999, VX-322, VEGF-trap, Imatinib mesylate, AzdO530, BMS-354825 (Dasatinib), SKI-606, CP-690, Tyrophistin AG-490, WHI-P154, WHI-P131, Sirolimus, Everolimus, AP23573, Fasudil hydrochloride, Flavopiridol, Seliciclib (CYC202, roscovitine), SNS-032, Ruboxistaurin, Pkc412, Bryostatin, KAI-9803, SF1126, VX-680, Azd1152, Arry-142886 (AZD-6244), SCIO-469, GW681323 (SB-681323), CC-401, CEP-1347, ENMD-2076, Semaxanib (SU5416), Sunitinib (SU11248) and Sorafenib (BAY 43-9006).

### Histone Deacetylase Inhibitors

Histone deacetylase inhibitors that may be used with the following invention include, but are not limited to, AN-9 (butyric acid prodrug), sodium phenylbutrate, valproic acid, FK-228 (cyclic depsipeptide), MGCD0103, MS-275 (benzamide), suberoylanilide hydroxamic acid (SAHA), and LAQ-824.

### Proteosome Inhibitors

Proteosomes are enzymes with a complex structure and function. They are found abundantly in all cells, both normal and cancerous, and are responsible for the degradation of all regulatory proteins. Proteosome inhibitors include those compounds capable of inhibiting the assembly and/or function of these complexes. An example of a proteosome inhibitor that may be used with the present invention includes, but is not limited to, bortezomib.

### Administration

In accordance with the present invention, the compounds of Formula I may be combined with one or more anti-cancer agents into a single formulation. The compounds of Formula I and the one or more anti-cancer agents may also be formulated and administered separately.

The compositions described herein can be provided as physiologically acceptable formulations using known techniques, and the formulations can be administered by standard routes. In general, analogs of 2-methoxyestradiol and the anti-cancer agent can be administered by topical, oral, rectal, nasal, inhalation or parenteral (e.g., intravenous, subcutaneous, intramuscular, intradermal, intraocular, intratracheal or epidural) routes. In addition, the compositions can be incorporated into polymers allowing for sustained release, the polymers being implanted in the vicinity of where delivery is desired, for example, at the site of a tumor or within or near the eye, or the polymers can be implanted, for example, subcutaneously or intramuscularly or delivered intravenously or intraperitoneally to result in systemic delivery of the analog of 2-methoxyestradiol and/or anti-cancer agent. Other formulations for controlled, prolonged release of therapeutic agents useful in the present invention are disclosed in U.S. Pat. No. 6,706,289, the disclosure of which is incorporated herein by reference.

Formulations contemplated as part of the present invention include nanoparticle formulations made by methods disclosed in U.S. patent application Ser. No. 10/392,403 (Publication No. 2004/0033267). By forming nanoparticles, the compositions disclosed herein are shown to have increased bioavailability. Preferably, the particles of the compounds of the present invention have an effective average particle size of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods well known to those of ordinary skill in the art.

The formulations in accordance with the present invention can be administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

The formulations include those suitable for oral, rectal, nasal, inhalation, topical (including dermal, transdermal, buccal and sublingual), vaginal, parenteral (including subcutaneous, intramuscular, intravenous, intradermal, intraocular, intratracheal, and epidural) or inhalation administration. The formulations can conveniently be presented in unit dosage form and can be prepared by conventional pharmaceutical techniques. Such techniques include the step of bringing into association the active ingredient(s) and a pharmaceutical carrier(s) or excipient(s). In general, the formulations are prepared by uniformly and intimately bringing into association the active ingredient(s) with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient or ingredients; as a powder or granules; as a solution or a suspension in an aqueous liquid or a non-aqueous liquid; or as an oil-in-water liquid emulsion or a water-in-oil emulsion, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface-active or dispersing agent. Molded tablets may be made by molding, in a suitable machine, a mixture of the powdered compound or compounds moistened with an inert liquid diluent. The tablets may optionally be coated or scored and may be formulated so as to provide a slow or controlled release of the active ingredient therein.

Formulations suitable for topical administration in the mouth include lozenges comprising the ingredients in a flavored base, usually sucrose and acacia or tragacanth; pastilles comprising the active ingredients in an inert base such as gelatin and glycerin, or sucrose and acacia; and mouthwashes comprising the ingredient to be administered in a suitable liquid carrier.

Formulations suitable for topical administration to the skin may be presented as ointments, creams, gels or pastes comprising the ingredient to be administered in a pharmaceutical acceptable carrier. In one embodiment the topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is taken; i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing, in addition to the active ingredient, ingredients such as carriers that are known in the art to be appropriate.

Formulations suitable for inhalation may be presented as mists, dusts, powders or spray formulations containing, in addition to the active ingredient, ingredients such as carriers that are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Formulations suitable for parenteral administration include particulate preparations of the anti-angiogenic agents, including, but not limited to, low-micron, or nanometer (e.g., less than 2000 nanometers, preferably less than 1000 nanometers, most preferably less than 500 nanometers, especially less than 75 nanometers in average cross section) sized particles, which particles are comprised of 2-methoxyestradiol analogs and/or one or more anti-cancer agents alone or in combination with accessory ingredients or in a polymer for sustained release. The formulations may be presented in unit-dose or multi-dose containers, for example, sealed ampules and vials, and may be stored in freeze-dried (lyophilized) conditions requiring only the addition of a sterile liquid carrier, for example, water for injections, immediately prior to use. Extemporaneous injection solutions and suspensions may be prepared from sterile powders, granules and tablets of the kinds previously described.

In one embodiment, one or more compounds of Formula I and one or more anti-cancer agents can be administered simultaneously. In another embodiment, one or more compounds of Formula I and one or more anti-cancer agents can be administered sequentially (e.g., 2ME2 analog dosage in the morning, anti-cancer agent dosage in the evening). Mixtures of more than one anti-cancer agent can, of course, be administered. Indeed, it is often desirable to use mixtures or sequential administrations of different anti-cancer agents to treat tumors, especially anti-cancer agents from the different classes mentioned above.

If the 2-methoxyestradiol analog formulation and the anti-cancer agent are to be administered sequentially, the amount of time between administration of the 2-methoxyestradiol analog formulation and the anti-cancer agent will depend upon factors such as the amount of time it takes the 2-methoxyestradiol analog formulation to be fully incorporated into the circulatory system of the host and the retention time of the 2-methoxyestradiol analog formulation in the host's body. In one embodiment, dosage formulations for 2-methoxyestradiol analogs are disclosed in U.S. patent application Ser. No. 11/288,989, filed Nov. 29, 2005, which is incorporated herein by reference in its entirety.

The anti-cancer agent is administered in a therapeutically effective amount. This amount will be determined on an individual basis and will be based, at least in part, on consideration of the host's size, the specific disease to be treated, the severity of the symptoms to be treated, the results sought, and other such considerations. An effective amount can be determined by one of ordinary skill in the art employing such factors and using no more than routine experimentation.

It should be understood that, in addition to the ingredients particularly mentioned above, the formulations of the present invention may include other agents conventional in the art having regard to the type of formulation in question, for example, those suitable for oral administration may include flavoring agents, and nanoparticle formulations (e.g. less than 2000 nanometers, preferably less than 1000 nanometers, most preferably less than 500 nanometers, especially less than 150 nanometers in average cross section) may include one or more than one excipient chosen to prevent particle agglomeration.

### Indications

The invention can be used to treat any disease characterized by abnormal cell proliferation and/or abnormal or undesirable angiogenesis. Such diseases include, but are not limited to, abnormal stimulation of endothelial cells (e.g., atherosclerosis); solid tumors; blood-borne tumors, such as leukemias; tumor metastasis; benign tumors, for example, hemangiomas, acoustic neuromas, neurofibromas, trachomas, and pyogenic granulomas; vascular malfunctions; abnormal wound healing; inflammatory and immune disorders; Bechet's disease; gout or gouty arthritis; abnormal angiogenesis accompanying: rheumatoid arthritis; skin diseases, such as psoriasis; diabetic retinopathy, and other ocular angiogenic diseases, such as retinopathy of prematurity (retrolental fibroplasia), macular degeneration, corneal graft rejection, neovascular glaucoma; liver diseases and Osler Webber Syndrome (Osler-Weber Rendu disease), epidemic keratoconjunctivitis, Vitamin A deficiency, contact lens overwear, atopic keratitis, superior limbic keratitis, pterygium keratitis sicca, Sjogren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi 's sarcoma, Mooren's ulcer, Terrien's marginal degeneration, marginal keratolysis, trauma, rheumatoid arthritis, systemic lupus, polyarteritis, Wegener's sarcoidosis, Scleritis, Steven-Johnson disease, pemphigoid, radial keratotomy, and corneal graph rejection.

Other diseases associated with neovascularization can be treated according to the present invention. Such diseases include, but are not limited to, sickle cell anemia, sarcoid, pseudoxanthoma elasticum, Paget's disease, vein occlusion, artery occlusion, carotid obstructive disease, chronic uveitis/vitritis, Lyme's disease, systemic lupus erythematosis, Eales' disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, and post-laser complications. Other diseases include, but are not limited to, diseases associated with rubeosis (neovascularization of the iris and the angle) and diseases caused by the abnormal proliferation of fibrovascular or fibrous tissue including all forms of proliferative vitreoretinopathy, whether or not associated with diabetes.

The present invention may also be used to treat angiogenesis-dependent cancers including, but not limited to, any one or combination of rhabdomyosarcoma, retinoblastoma, Ewing's sarcoma, neuroblastoma, and osteosarcoma. Other angiogenesis-dependent cancers treatable with the present invention include, but are not limited to, breast cancer, prostate cancer, renal cell cancer, brain cancer, ovarian cancer, colon cancer, bladder cancer, pancreatic cancer, stomach cancer, esophageal cancer, cutaneous melanoma, liver cancer, small cell and non-small cell lung cancer, testicular cancer, kidney cancer, bladder cancer, cervical cancer, lymphoma, parathyroid cancer, penile cancer, rectal cancer, small intestine cancer, thyroid cancer, uterine cancer, Hodgkin's lymphoma, non-Hodgkin's lymphoma, lip cancer, oral cancer, skin cancer, leukemia or multiple myeloma.

### Pharmaceutical Preparations

Also contemplated by the present invention are implants or other devices comprised of the compounds or drugs of Formula I and one or more anti-cancer agents, or prodrugs thereof, or other compounds included by reference where the drug or prodrug is formulated in a biodegradable or non-biodegradable polymer for sustained release. Non-biodegradable polymers release the drug in a controlled fashion through physical or mechanical processes without the polymer itself being degraded. Biodegradable polymers are designed to gradually be hydrolyzed or solubilized by natural processes in the body, allowing gradual release of the admixed drug or prodrug. The drug or prodrug can be chemically linked to the polymer or can be incorporated into the polymer by admixture. Both biodegradable and non-biodegradable polymers and the process by which drugs are incorporated into the polymers for controlled release are well known to those skilled in the art. Examples of such polymers can be found in many references, such as Brem et al., J. Neurosurg (1991) 74:441-446. These implants or devices can be implanted in the vicinity where delivery is desired, for example, at the site of a tumor or a stenosis, or can be introduced so as to result in systemic delivery of the agent.

Because anything not formed in the body as a natural component may elicit extreme and unexpected responses, such as blood vessel closure due to thrombus formation or spasm, and because damage to blood vessels by the act of insertion of a vascular stent may be extreme and unduly injurious to the blood vessel surface, it is prudent to protect against such events. Restenosis is a re-narrowing or blockage of an artery at the same site where treatment, such as an angioplasty or stent procedure, has already taken place. If restenosis occurs within a stent that has been placed in an artery, which is known as “in-stent restenosis,” the end result being a narrowing in the artery caused by a build-up of substances that may eventually block the flow of blood. The compounds that are part of the present invention are especially useful to coat vascular stents to prevent restenosis. The coating should preferably be a biodegradable or non-biodegradable polymer that allows for a slow release of the composition of the present invention thereby preventing the restenosis event.

The present invention also relates to conjugated prodrugs and uses thereof. More particularly, the invention relates to conjugates of steroid compounds, such as compounds of Formula I, and the use of such conjugates in the prophylaxis or treatment of conditions associated with enhanced angiogenesis or accelerated cell division, such as cancer, and inflammatory conditions, such as asthma and rheumatoid arthritis and hyperproliferative skin disorders including psoriasis. The invention also relates to compositions including the prodrugs of the present invention and methods of synthesizing the prodrugs.

In one aspect, the present invention provides a conjugated prodrug of an estradiol analog compound, preferably compounds of Formula I conjugated to a biological active modifying agent.

Alternatively, the conjugated prodrug according to the present invention includes the compounds of Formula I conjugated to a peptide moiety.

The incorporation of an estradiol compound, such as the compounds of Formula I into a disease-dependently activated pro-drug enables significant improvement of potency and selectivity of this anti-cancer and anti-inflammatory agent.

A person skilled in the art will be able by reference to standard texts, such as Remington's Pharmaceutical Sciences 17th edition, to determine how the formulations are to be made and how these may be administered.

In a further aspect of the present invention there is provided use of compounds of Formula I, or prodrugs thereof, in combination with one or more anti-cancer agents according to the present invention for the preparation of a medicament for the prophylaxis or treatment of conditions associated with angiogenesis or accelerated cell division or inflammation.

In a still further aspect of the present invention there is provided a method of prophylaxis or treatment of a condition associated with angiogenesis or accelerated or increased amounts of cell division, hypertrophic growth or inflammation, said method including administering to a patient in need of such prophylaxis or treatment an effective amount of compounds of Formula I, or prodrugs thereof, in combination with an anti-cancer agent according to the present invention, as described herein. It should be understood that prophylaxis or treatment of said condition includes amelioration of said condition.

Pharmaceutically acceptable salts of the compounds of the Formula I or the prodrugs thereof, can be prepared in any conventional manner, for example from the free base and acid. In vivo hydrolysable esters, amides and carbamates and other acceptable prodrugs of Formula I can be prepared in any conventional manner.

100% pure isomers are contemplated by this invention; however a stereochemical isomer (labeled as α or β, or as R or S) may be a mixture of both in any ratio, where it is chemically possible by one skilled in the art. Also contemplated by this invention are both classical and non-classical bioisosteric atom and substituent replacements, such as are described by Patani and Lavoie (“Bio-isosterism: a rational approach in drug design” Chem. Rev. (1996) pp. 3147-3176) and are well known to one skilled in the art. Such bioisosteric replacements include, for example, but are not limited to, substitution of ═S or ═NH for ═O.

A particularly useful formulation in the present invention is a nanoparticulate liquid suspension of 2-methoxyestradiol disclosed in U.S. patent application Ser. No. 10/392,403, filed Mar. 20, 2003 (the disclosure of which is incorporated herein by reference). This formulation is available from EntreMed, Inc., Rockville, Md., under the designation Panzem® NCD.

### Estradiol Derivative Synthesis

Known compounds that are used in accordance with the invention and precursors to novel compounds according to the invention can be purchased, e.g., from Sigma Chemical Co., Steraloids or Research Plus. Other compounds according to the invention can be synthesized according to known methods from publicly available precursors.

The chemical synthesis of estradiol has been described (Eder, V. et al., Ber (1976) 109:2948; Oppolzer, D. A. and Roberts D. A. Helv. Chim. Acta. (1980) 63:1703). The synthetic pathways used to prepare some of the derivatives of the present invention are based on modified published literature procedures for estradiol derivatives (Trembley et al., Bioorganic & Med. Chem. (1995) 3:505-523; Fevig et al., J. Org. Chem., (1987) 52:247-251; Gonzalez et al., Steroids (1982) 40:171-187; Trembley et al., Synthetic Communications (1995), 25:2483-2495; Newkome et al., J. Org. Chem. (1966) 31:677-681; Corey et al. Tetrahedron Lett. (1976) 3-6; Corey et al., Tetrahedron Lett., (1976) 3667-3668; and German Patent No. 2757157 (1977)).

The 2-position of estradiol can be modified according to known methods, such as those described in U.S. Pat. No. 6,136,992 (incorporated herein by reference); Siya Ram et al., 2-Alkoxy estradiols and derivatives thereof (incorporated herein by reference); Cushman et al., J. Med. Chem. (1995) 38:2041-2049; Cushman, et. al., J. Med. Chem. (1997) 40:2323-2334; U.S. Pat. No. 6,448,419 (2002); Paaren et al., Wang et al., Synthetic Comm (1998) 28:4431, Cushman et al., J. Med. Chem. (1995) 38:2041, Cushman et al., J. Med. Chem. (2002) 45: 4748), or other publications, incorporated herein by reference.

The synthetic routes for the series of analogs disclosed by this invention can be synthesized according to known methods, such as those described in U.S. patent application Ser. No. 11/077,977, filed Mar. 11, 2005 (incorporated herein by reference).

### Evaluation of 2-Methoxyestradiol Analogs in Combination With Another Anti-Cancer Agent

A number of methods exist in the art for assessing the effectiveness of drug combinations including, but not limited to, the isobologram introduced by Loewe (Pharmacol. Rev. (1957) 9:237-242), determination of the fractional inhibitory concentration index (FICI) (Webb, J. Enzymes and Metabolic Inhibitors, Vol. 1, pp. 66-79 and 487-512, Academic Press, New York, 1963), or determination of the combination index (CI) (Chou T. and Talalay P. Adv Enzyme Regul., 22:27-55 (1984)). In one embodiment, determination of the combination index is used to assess the effectiveness of 2-methoxyestradiol analog and anti-cancer agent combinations.

Prior to analysis by any of the above methods, a dose-effect curve for each drug alone and in at least one fixed ratio combination must be determined. The dose-effective curve can be determined from experiments carried out in an appropriate in vitro model. Methods and in vitro models are well known in the art and the most appropriate method and model can determined for a given experimental design by one of ordinary skill in the art. Examples of relevant cell lines for use in such experiments include, but are not limited to, MDA-MBA-231 (breast cancer), U87-MG (glioblastoma), PC3 (prostate cancer) and HUVEC (human umbilical vein endothelial cells).

The above methods allows one of skill in the art to determine optimal doses for each individual drug and drug combination and whether a particular combination can be expected to have an antagonistic effect, a synergistic effect, or an additive effect. This information can then be used to select the most promising combinations and direct further validation in one or more in vivo models.

### EXPERIMENTAL EXAMPLES

The compositions and methods are further illustrated by the following non-limiting examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention.

### Experimental Data

The following Examples refer to compounds of the following general Formula I:

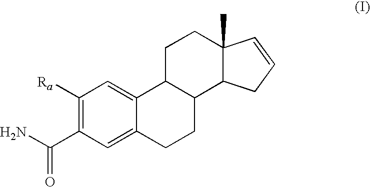

wherein Rₐ is selected from —OCH₃, —OCH₂CH₃ or —C≡C—CH₃ administered in combination with anti-cancer agents. Preferred species from the foregoing genus that are useful in the present invention include, but are not limited to, the compounds of Table I.

TABLE I

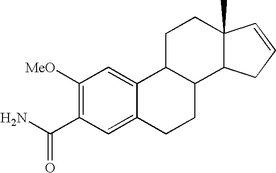

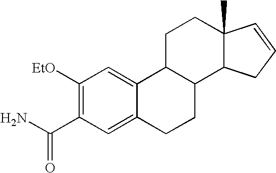

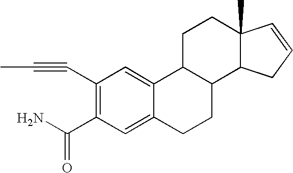

Each of the foregoing compounds from Table I is found to have anti-mitotic properties, anti-angiogenic properties, anti-tumor properties or combinations thereof.

### Example 1

### Protocol for Determination of 2ME2 Analog+Anti-Cancer Agent Combination Index (CI)

U87-MG human glioblastoma cells can be maintained in vitro in DMEM supplemented with 5% FBS, 2 mM glutamine, 1 mM sodium pyruvate, MEM vitamins and NEAA at 37° C. and 5% CO₂. For U87-MG proliferation assays, cells will need to be plated in a 96-well plate at 5×10³ cells per well and incubated at 37° C. overnight. At 24 hours, the media is then aspirated and 2-methoxyestradiol analog or anti-cancer agent will be administered to the cells at multiple doses. Proliferation can then be assessed 48 hours after application of the drug by WST-1 (Francoeur et al. “A novel cell proliferation and cytotoxicity assay,” Biochemica; 3:19-25 (1996)). Dose response curves are then constructed using suitable software, such as Table Curve 2D software (SPSS, Inc., Chicago, Ill.) and used to determine each drug's IC₅₀.

The 2-methoxyestradiol analog and anti-cancer agent are then administered in combination at fixed ratios. More than one fixed ratio can be used. In general, an equipotent ratio (e.g. the ratio of their IC₅₀ values) is a good initial ratio with which to work. Next, a concentrated mixture of the two drugs is made (e.g. 4-fold their IC₅₀ values) and serial dilutions are made (e.g. 2-fold dilutions resulting in 2-fold, 1-fold, 0.5-fold, 0.25-fold, 0.125-fold concentrations). The cells are maintained and the drug combinations will be administered under the same conditions used above for each drug individually. Each dose is usually administered at least in quadruplicate. Cell proliferation is then determined as above using WST-1. The resulting dose response curves can then be averaged to create a single composite dose response curve for each fixed ratio combination analyzed. The fraction affected [(1−absorbance at 490 nm of treatment group−blank)/(absorbance at 490 nm of untreated control−blank)] can then be calculated and entered into CalcuSyn software (Biosoft, Cambridge, UK). CalcuSyn software is based on the median effect method of Chou and Talalay (Chou and Talalay, 1983), and is used to determine the combination index, wherein a combination index of <1, a combination index=1, and a combination index >1, indicates synergy, additivity, and antagonism, respectfully. This assay can be used to screen 2ME2 analogs and anti-cancer combinations for their therapeutic potential and guide further validation in vivo.

### Example 2

### Determination of 2ME2 Analog+Sutent® Combination Index (CI)

The combination index of Sutent® and ENMD-1198 (a 2-methoxyestradiol analog) at various concentrations was determined using the protocol of Example 1. Caki-1 cells, a human renal cell line, were used in place of U87-MG human glioblastoma cells. The calculated CI values at each concentration are provided in Table 2.

TABLE 2
Sutent: ENMD-1198
(Caki-1 Cells)

| | 1198 | |
|---|---|---|
| Sutent (nM) | (nM) | CI |
| 672.5 | 91.1 | 0.92 |
| 1345 | 182.1 | 1.12 |
| 2690 | 364.3 | 0.57 |
| 5380 | 728.5 | 0.52 |
| 10760 | 1457 | 0.85 |
| 21520 | 2914 | 0.79 |
| 43040 | 5828 | 1.21 |

### Example 3

### Determination of 2-methoxyestradiol Analog+Rapamyacin Combination Index (CI)

The combination index of Rapamyacin and ENMD-1198 at various concentrations was determined using the protocol of Example 1. The calculated CI values at each concentration are provided in Table 3.

TABLE 3
Rapamycin: ENMD-1198
(U87-MG Cells)

| Rapamycin | 1198 | |
|---|---|---|
| (nM) | (nM) | CI |
| 49.6 | 4.9 | 0.23 |
| 99.3 | 9.8 | 0.38 |
| 198.5 | 19.7 | 0.73 |
| 397 | 39.3 | 0.74 |
| 794 | 78.6 | 0.65 |
| 1588 | 157.3 | 0.26 |
| 3176 | 314.6 | 0.12 |

We claim:

1. A composition for treating diseases associated with undesirable and/or excessive angiogenesis and/or undesirable cell proliferation comprising;
a) one or more compounds selected from a group comprising: 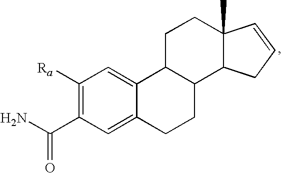
wherein Rₐ is —OCH₃, —OCH₂CH₃ or —C≡C—CH₃;
b) one or more anti-cancer agents; and
c) a pharmaceutically acceptable carrier, diluent, or excipient.

2. A method of treating diseases associated with undesirable and/or excessive angiogenesis and/or undesirable cell proliferation comprising administering simultaneously to an animal or human;
a) a first composition comprising one or more therapeutic agents selected from a group comprising: 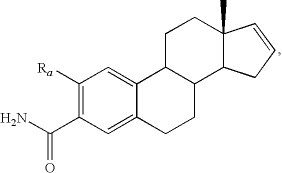
wherein Rₐ is —OCH₃, —OCH₂CH₃ or —C≡C—CH₃; and
b) one or more anti-cancer agents.

3. The method of claim 2, wherein the first composition and the one or more anti-cancer agents can be administered in a single formulation or in two or more separate formulations.

4. A method for treating diseases associated with undesirable and/or excessive angiogenesis and/or undesirable cell proliferation comprising administering sequentially to an animal or human;
a) a first composition comprising one or more therapeutic agents selected from a group comprising: 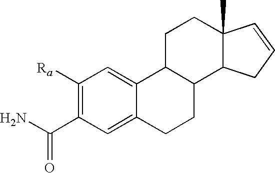
wherein Rₐ is —OCH₃, —OCH₂CH₃ or —C≡C—CH₃; and
b) one or more anti-cancer agents.
